(19) United States
(12) Patent Application Publication
(10) Pub. No.: US 2007/0042390 A1
(43) Pub. Date: Feb. 22, 2007

(54) Method and device for critical dimension detection by molecular binding

(75) Inventor: Peter Borden, San Mateo, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Applied Materials, Inc. A Delaware corporation, Santa Clara, CA (US)

(21) Appl. No.: 11/207,351

(22) Filed: Aug. 19, 2005

(51) Int. Cl. C12Q 1/68; G01N 33/53; G06K 9/00; G06F 19/00

(52) U.S. Cl. 435/6; 435/7.1; 382/128; 702/19

(57) ABSTRACT

Critical Dimension (CD) of features on a semiconductor substrate may be indicated utilizing the site-specific binding properties of organic or biological molecules. In accordance with one embodiment of the present invention, a fluorescent tagged organic molecule is fabricated having a length corresponding to the desired CD. The semiconductor substrate is exposed to a solution containing the organic molecule. The solution is then removed and the structure analyzed for the presence of the fluorescent tag, indicating a feature having the desired CD. Fluorescent tagged biological molecules of known size such as peptides or proteins, or nucleic acids such as DNA or RNA, may also be employed for CD measurement. Alternatively, a CD marker molecule may be designed to exhibit preferential binding, such that it fails to bind to the substrate in instances of incomplete resist development or etching.

### BACKGROUND OF THE INVENTION

Semiconductor device geometries have dramatically decreased in size since such devices were first introduced several decades ago. As device geometries have become more dense, reductions in the spacing between device elements has occurred. The minimum linewidths achieved using semiconductor lithography systems, sometimes referred to as a critical dimension (CD) have decreased over time.

Lithography or photolithography generally refers to processes for transferring patterns between a mask and a semiconductor substrate. In lithography processes for semiconductor device fabrication, a silicon substrate is uniformly coated with a photosensitive material, referred to as a photoresist, in a cluster tool. A scanner/stepper tool selectively exposes the photoresist to some form of electromagnetic radiation to generate a circuit pattern corresponding to an individual layer of the integrated circuit (IC) device to be formed on the substrate surface. Generally, the photoresist film is selectively exposed using a mask layer that preferentially blocks a portion of the incident radiation. The portions of the photoresist film that are exposed to the incident radiation become more or less soluble depending on the type of photoresist that is utilized. A developing stage dissolves the more soluble regions of the photoresist film, producing a patterned photoresist layer corresponding to the mask layer used in the exposure process.

Once lithographic processing has been performed, it is important to measure the resulting feature dimensions, such as CD. Such measurements are important to ensure that patterns have been exposed properly in photolithography. Measurements are made on either photoresist layers or on features after the photoresist has been removed. For example, transistor gates are over-etched, and it is important to know the width of the gate lines, as this determines the speed of the transistor. In another example, a measurement is made on photoresist directly after development, so that the resist can be reworked should a dimensional tolerance error be found.

Two methods have conventionally been employed to measure critical dimension. In scanning electron microscopy (SEM), an electron microscope images a feature. Analysis of the image provides a measure of the CD.

An alternative conventional technique for measuring CD is optical critical dimension (OCD), also known as Optical Digital Profilometry (ODP). In OCD, light is scattered from an array of lines. Analysis of the resulting interference pattern provides a measure of the average CD over the illuminated area.

However, the conventional OCD also suffers from a number of disadvantages. One disadvantage is accuracy. Specifically, OCD has a limit of resolution of a few tens of angstroms, again insufficient for measuring CD for features of 45 nm or less.

In addition, OCD requires a relatively large measurement area of several tens of microns, since its accuracy relies on averaging light scattering over a large number of lines. It also requires complex models that must be tailored to the particular pattern of lines that is to be employed for the measurement.

Another potential disadvantage associated with OCD is reliability. Specifically, OCD systems are relatively complex and unreliable. Failure in an OCD module can idle an extremely expensive track lithography cell module.

Still another potential disadvantage associated with OCD is limited mapping capability. OCD typically generates only one profile per wafer. A detailed wafer map enabling feedback to the stepper to allow control of the exposure over the full wafer, would thus require a considerable time to generate.

Finally, OCD requires the compiling of reference libraries for each structure that is to be measured. Structures can change with part types, so large libraries are needed for the OCD to function with all products that run through the litho cell. These large libraries are expensive and time consuming to create and store.

Other implementations of OCD perform complex computations after measurement. Such complex computations require an expensive processor, which may still be too slow to provide a wafer mapping capability at a throughput consistent with lithographic processing requirements.

In many applications, such as measuring CD after photoresist development on a track system, it is desirable to have a simple CD measurement that does not require a large test structure or a complex computer model. Therefore, there is a need in the art for improved systems and methods for measuring critical dimension during processing.

### BRIEF SUMMARY OF THE INVENTION

Critical Dimension (CD) in a semiconductor structure may be accurately measured utilizing site-specific binding properties of organic molecules or biological molecules. In accordance with one embodiment of the present invention, a fluorescent tagged organic molecule is fabricated having a length corresponding to the desired CD. The semiconductor device is exposed to a solution containing the organic molecule. The solution is then removed, and the structure is analyzed for the presence of the fluorescent tag indicating a feature having the CD. Fluorescent tagged biological molecules of known size, such as peptides or proteins, or nucleic acids such as DNA or RNA, may also be employed for CD measurement purposes according to embodiments of the present invention.

An embodiment of a method in accordance with the present invention for providing information regarding a feature on a substrate, comprises, providing a substrate having a feature, exposing the substrate to a molecule selectively binding to the feature, and detecting the molecule bound to the feature.

An alternative embodiment of a method in accordance with the present invention, comprises, identifying a critical dimension of a feature on a substrate, and fabricating a molecule having a property allowing the molecule to selectively bind to the feature.

An embodiment of a composition in accordance with the present invention for indicating conformity of a substrate feature to a critical dimension (CD), comprises, a solvent and a molecule having a fluorescent tag, a length corresponding to a critical dimension, and a site exhibiting binding affinity with the feature.

An embodiment of an apparatus in accordance with the present invention for detecting a feature on a semiconductor workpiece, comprises, walls enclosing a chamber housing a substrate support and a drain, and a fluid inlet configured to receive at least one of diluent from a first reservoir and a marker molecule solution from a second reservoir. A radiation source is in electromagnetic communication with the chamber and configured to irradiate a substrate positioned on the support with excitation radiation. A detector is in electromagnetic communication with the chamber through a filter and is configured to sense radiation emitted from the substrate in response to exposure to the excitation radiation.

These and other embodiments of the invention along with many of its advantages and features are described in more detail in conjunction with the text below and attached figures.

### BRIEF DESCRIPTION OF THE DRAWINGS

FIG. 1 is a plan view of one embodiment of a track lithography tool according to one embodiment of the present invention.

FIG. 2 is a flowchart illustrating a processing sequence for a semiconductor substrate according to one embodiment of the present invention.

FIG. 3 is a simplified flow chart illustrating a method in accordance with an embodiment of the present invention.

FIG. 4A is a simplified cross-sectional view of protein molecules binding to a repeating structure in accordance with one embodiment of the present invention.

FIG. 4B is a simplified cross-sectional view of protein molecules binding to a single structure in accordance with another embodiment of the present invention.

FIG. 5A is a simplified schematic view of an example of the use of DNA as a marker for critical dimension for a repeating structure in accordance with an embodiment of the present invention.

FIG. 5B is a simplified schematic view of an example of the use of DNA as a marker for critical dimension of an isolated structure in accordance with an embodiment of the present invention.

FIGS. 6A-B is a simplified schematic views of an example of the use of a tagged molecule to determine the extent of development of photoresist.

FIGS. 7A-B shows a simplified schematic views of an example of the use of a tagged molecule to determine the extent of etching.

FIG. 8 shows a simplified schematic view of an embodiment of an apparatus in accordance with the present invention for detecting features on a workpiece.

### DETAILED DESCRIPTION OF THE INVENTION

According to various embodiments, techniques related to the field of measurement of extremely small distances are provided. One particular embodiment in accordance with the present invention relates to methods for measurement of critical dimensions (CD) of features such as fine lines used in the manufacture of integrated circuits. Merely by way of example, the method and apparatus have been applied to processing a semiconductor workpiece. But it would be recognized that the invention has a much broader range of applicability.

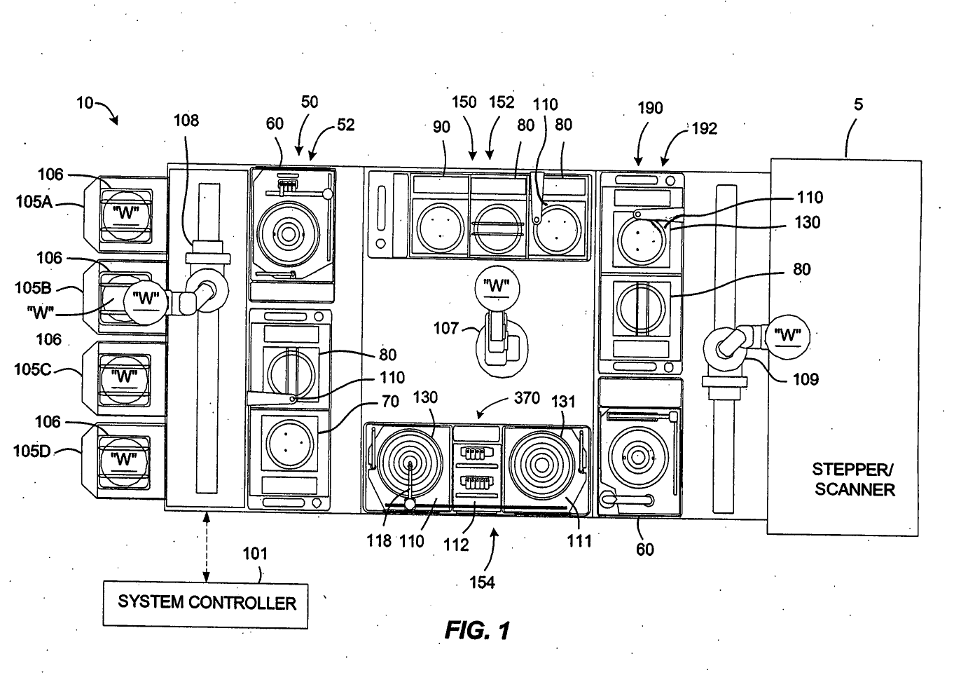

FIG. 1 is a plan view of one embodiment of a track lithography tool 10 in which the developer endpoint detection system of the present invention may be used. One embodiment of the track lithography tool 10, as illustrated in FIG. 1, contains a front end module (sometimes referred to as a factory interface) 50, a central module 150, and a rear module (sometimes referred to as a scanner interface) 190. The front end module 50 generally contains one or more pod assemblies or FOUPS 105 (e.g., items 105A-D), a front end robot 108, and a front end processing rack 52. The central module 150 will generally contain a first central processing rack 152, a second central processing rack 154, and a central robot 107. The rear module 190 will generally contain a rear processing rack 192 and a back end robot 109. In one embodiment, the track lithography tool 10 contains: a front end robot 108 adapted to access processing modules in the front end processing rack 52; a central robot 107 that is adapted to access processing modules in the front end processing rack 52, the first central processing rack 152, the second central processing rack 154 and/or the rear processing rack 192; and a back end robot 109 that is adapted to access processing modules in the rear processing rack 192 and in some cases exchange substrates with a stepper/scanner 5. In one embodiment, a shuttle robot is adapted to transfer substrates between two or more adjacent processing modules retained in one or more processing racks (e.g., front end processing rack 52, first central processing rack 152, etc.). In one embodiment, a front end enclosure is used to control the environment around the front end robot 108 and between pods assemblies 105 and front end processing rack 52.

FIG. 1 also contains more detail of possible process chamber configurations found in aspects of the invention. For example, the front end module 50 generally contains one or more pod assemblies or FOUPs 105, a front end robot 108 and a front end processing rack 52. The one or more pod assemblies 105, are generally adapted to accept one or more cassettes 106 that may contain one or more substrates “W”, or wafers, that are to be processed in the track lithography tool 10. The front end processing rack 52 contains multiple processing modules (e.g., bake plate 90, chill plate 80, etc.) that are adapted to perform the various processing stages found in the substrate processing sequence. In one embodiment, the front end robot 108 is adapted to transfer substrates between a cassette mounted in a pod assembly 105 and between the one or more processing modules retained in the front end processing rack 52.

The central module 150 generally contains a central robot 107, a first central processing rack 152 and a second central processing rack 154. The first central processing rack 152 and a second central processing rack 154 contain various processing modules (e.g., coater/developer module with shared dispense 370, bake module 90, chill plate 80, etc.) that are adapted to perform the various processing stages found in the substrate processing sequence. In one embodiment, the central robot 107 is adapted to transfer substrates between the front end processing rack 52, the first central processing rack 152, the second central processing rack 154 and/or the rear processing rack 192. In one aspect, the central robot 107 is positioned in a central location between the first central processing rack 152 and a second central processing rack 154 of the central module 150.

The rear module 190 generally contains a rear robot 109 and a rear processing rack 192. The rear processing rack 192 generally contains processing modules (e.g., coater/developer module 60, bake module 90, chill plate 80, etc.) that are adapted to perform the various processing stages found in the substrate processing sequence. In one embodiment, the rear robot 109 is adapted to transfer substrates between the rear processing rack 190 and a stepper/scanner 5. The stepper/scanner 5, which may be purchased from Canon USA, Inc. of San Jose, Calif., Nikon Precision Inc. of Belmont, Calif., or ASML US, Inc. of Tempe Ariz., is a lithographic projection apparatus used, for example, in the manufacture of integrated circuits (ICs). The scanner/stepper tool 5 exposes a photosensitive material (resist), deposited on the substrate in the cluster tool, to some form of electromagnetic or electron or ion beam radiation to generate a circuit pattern corresponding to an individual layer of the integrated circuit (IC) device to be formed on the substrate surface.

In one embodiment, a system controller 101 is used to control all of the components and processes performed in the cluster tool 10. The controller 101, is generally adapted to communicate with the stepper/scanner 5, monitor and control aspects of the processes performed in the cluster tool 10, and is adapted to control all aspects of the complete substrate processing sequence. The controller 101, which is typically a microprocessor-based controller, is configured to receive inputs from a user and/or various sensors in one of the processing chambers and appropriately control the processing chamber components in accordance with the various inputs and software instructions retained in the controller's memory. The controller 101 generally contains memory and a CPU (not shown) which are utilized by the controller to retain various programs, process the programs, and execute the programs when necessary. The memory (not shown) is connected to the CPU, and may be one or more of a readily available memory, such as random access memory (RAM), read only memory (ROM), floppy disk, hard disk, or any other form of digital storage, local or remote. Software instructions and data can be coded and stored within the memory for instructing the CPU. The support circuits (not shown) are also connected to the CPU for supporting the processor in a conventional manner. The support circuits may include cache, power supplies, clock circuits, input/output circuitry, subsystems, and the like all well known in the art. A program (or computer instructions) readable by the controller 101 determines which tasks are performable in the processing chamber(s). Preferably, the program is software readable by the controller 101 and includes instructions to monitor and control the process based on defined rules and input data.

FIG. 1 further illustrates a coater/developer module with a shared dispense 370 mounted in the second central processing rack 154, that may adapted to perform a photoresist coat stage or a develop stage in both of the process chambers 110 and 111. This configuration is advantageous since it allows some of the common components found in the two process chambers 110 and 111 to be shared thus reducing the system cost, complexity and tool footprint. As illustrated in FIG. 1 and described in more detail below, two spin chucks 130 and 131 are provided in processing chambers 110 and 111, respectively. A shared central fluid dispense bank 112 is positioned between the two processing chambers and dispense arm assembly 118 is able to select nozzles from the central fluid dispense bank and serve both spin chucks. Central robot 107 as illustrated in FIG. 1 is able to access both processing chambers 110 and 111 independently.

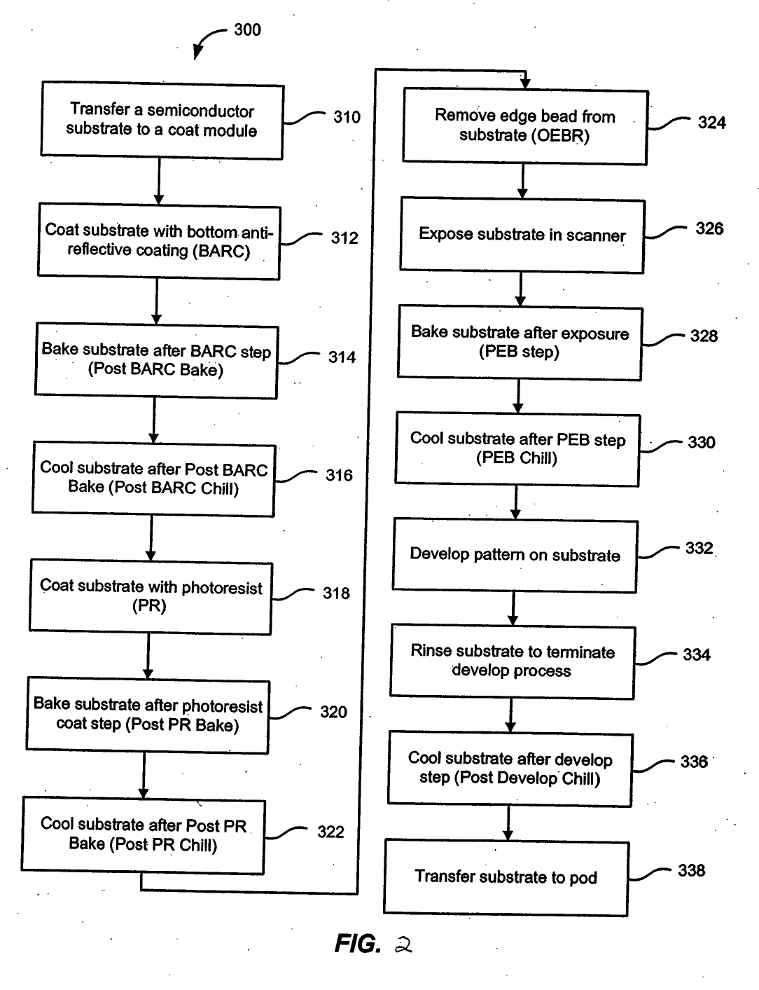

FIG. 2 is a flowchart illustrating a processing sequence for a semiconductor substrate according to one embodiment of the present invention. FIG. 2 illustrates one embodiment of a series of method stages 300 that may be used to deposit, expose and develop a photoresist material layer formed on a substrate surface. The lithographic process may generally contain the following: a transfer substrate to coat module stage 310, a bottom anti-reflective coating (BARC) coat stage 312, a post BARC bake stage 314, a post BARC chill stage 316, a photoresist coat stage 318, a post photoresist bake stage 320, a post photoresist chill stage 322, an optical edge bead removal (OEBR) stage 324, an exposure stage 326, a post exposure bake (PEB) stage 328, a post exposure bake chill stage 330, a develop stage 332, a rinse stage 334, a cool stage 336, and a transfer substrate to pod stage 338. In other embodiments, the sequence of the method stages 300 may be rearranged, altered, one or more stages may be removed, or two or more stages may be combined into a single stage with out varying from the basic scope of the invention.

In stage 310, a semiconductor substrate is transferred to a coat module. Referring to FIG. 1, the stage of transferring the substrate to the coat module 310 is generally defined as the process of having the front end robot 108 remove a substrate from a cassette 106 resting in one of the pod assemblies 105. A cassette 106, containing one or more substrates “W”, is placed on the pod assembly 105 by the user or some external device (not shown) so that the substrates can be processed in the cluster tool 10 by a user-defined substrate processing sequence controlled by software retained in the system controller 101.

The BARC coat stage 310 is a stage used to deposit an organic material over a surface of the substrate. The BARC layer is typically an organic coating that is applied onto the substrate prior to the photoresist layer to absorb light that otherwise would be reflected from the surface of the substrate back into the resist during the exposure stage 326 performed in the stepper/scanner 5. If these reflections are not prevented, standing waves will be established in the resist layer, which cause feature size to vary from one location to another depending on the local thickness of the resist layer. The BARC layer may also be used to level (or planarize) the substrate surface topography, which is generally present after completing multiple electronic device fabrication stages. The BARC material fills around and over the features to create a flatter surface for photoresist application and reduces local variations in resist thickness. The BARC coat stage 310 is typically performed using a conventional spin-on resist dispense process in which an amount of the BARC material is deposited on the surface of the substrate while the substrate is being rotated which causes a solvent in the BARC material to evaporate and thus causes the material properties of the deposited BARC material to change. The air flow and exhaust flow rate in the BARC processing chamber is often controlled to control the solvent vaporization process and the properties of the layer formed on the substrate surface.

The post BARC bake stage 314, is a stage used to assure that all of the solvent is removed from the deposited BARC layer in the BARC coat stage 312, and in some cases to promote adhesion of the BARC layer to the surface of the substrate. The temperature of the post BARC bake stage 314 is dependent on the type of BARC material deposited on the surface of the substrate, but will generally be less than about 250° C. The time required to complete the post BARC bake stage 314 will depend on the temperature of the substrate during the post BARC bake stage, but will generally be less than about 60 seconds.

The post BARC chill stage 316, is a stage used to control and assure that the time the substrate is above ambient temperature is consistent so that every substrate sees the same time-temperature profile and thus process variability is minimized. Variations in the BARC process time-temperature profile, which is a component of a substrates wafer history, can have an effect on the properties of the deposited film layer and thus is often controlled to minimize process variability. The post BARC chill stage 316, is typically used to cool the substrate after the post BARC bake stage 314 to a temperature at or near ambient temperature. The time required to complete the post BARC chill stage 316 will depend on the temperature of the substrate exiting the post BARC bake stage, but will generally be less than about 30 seconds.

The photoresist coat stage 318, is a stage used to deposit a photoresist layer over a surface of the substrate. The photoresist layer deposited during the photoresist coat stage 318 is typically a light sensitive organic coating that are applied onto the substrate and is later exposed in the stepper/scanner 5 to form the patterned features on the surface of the substrate. The photoresist coat stage 318 is a typically performed using conventional spin-on resist dispense process in which an amount of the photoresist material is deposited on the surface of the substrate while the substrate is being rotated which causes a solvent in the photoresist material to evaporate and thus causes the material properties of the deposited photoresist layer to change. The air flow and exhaust flow rate in the photoresist processing chamber is controlled to control the solvent vaporization process and the properties of the layer formed on the substrate surface. In some cases it may be necessary to control the partial pressure of the solvent over the substrate surface to control the vaporization of the solvent from the resist during the photoresist coat stage by controlling the exhaust flow rate and/or by injecting a solvent near the substrate surface. Referring to FIG. 1, in an exemplary photoresist coating process, the substrate is first positioned on wafer chuck 131 in coater/developer module 370. A motor rotates the wafer chuck 131 and substrate while the photoresist is dispensed onto the center of the substrate. The rotation imparts an angular torque onto the photoresist, which forces the photoresist out in a radial direction, to ultimately covering the substrate.

The post photoresist bake stage 320, is a stage used to assure that all of the solvent is removed from the deposited photoresist layer in the photoresist coat stage 318, and in some cases to promote adhesion of the photoresist layer to the BARC layer. The temperature of the post photoresist bake stage 320 is dependent on the type of photoresist material deposited on the surface of the substrate, but will generally be less than about 250° C. The time required to complete the post photoresist bake stage 320 will depend on the temperature of the substrate during the post photoresist bake stage, but will generally be less than about 60 seconds.

The post photoresist chill stage 322, is a stage used to control the time the substrate is at a temperature above ambient temperature so that every substrate sees the same time-temperature profile and thus process variability is minimized. Variations in the time-temperature profile can have an effect on properties of the deposited film layer and thus is often controlled to minimize process variability. The temperature of the post photoresist chill stage 322, is thus used to cool the substrate after the post photoresist bake stage 320 to a temperature at or near ambient temperature. The time required to complete the post photoresist chill stage 322 will depend on the temperature of the substrate exiting the post photoresist bake stage, but will generally be less than about 30 seconds.

The optical edge bead removal (OEBR) stage 324, is a process used to expose the deposited light sensitive photoresist layer(s), such as, the layers formed during the photoresist coat stage 318 and the BARC layer formed during the BARC coat stage 312, to a radiation source (not shown) so that either or both layers can be removed from the edge of the substrate and the edge exclusion of the deposited layers can be more uniformly controlled. The wavelength and intensity of the radiation used to expose the surface of the substrate will depend on the type of BARC and photoresist layers deposited on the surface of the substrate. An OEBR tool can be purchased, for example, from USHIO America, Inc. Cypress, Calif.

The exposure stage 326, is a lithographic projection stage applied by a lithographic projection apparatus (e.g., stepper scanner 5) to form a pattern which is used to manufacture integrated circuits (ICs). The exposure stage 326 forms a circuit pattern corresponding to an individual layer of the integrated circuit (IC) device on the substrate surface, by exposing the photosensitive materials, such as, the photoresist layer formed during the photoresist coat stage 318 and the BARC layer formed during the BARC coat stage 312 of some form of electromagnetic radiation.

The post exposure bake (PEB) stage 328, is a stage used to heat a substrate immediately after the exposure stage 326 in order to stimulate diffusion of the photoactive compound(s) and reduce the effects of standing waves in the resist layer. For a chemically amplified resist, the PEB stage also causes a catalyzed chemical reaction that changes the solubility of the resist. The control of the temperature during the PEB is typically critical to critical dimension (CD) control. The temperature of the PEB stage 328 is dependent on the type of photoresist material deposited on the surface of the substrate, but will generally be less than about 250° C. The time required to complete the PEB stage 328 will depend on the temperature of the substrate during the PEB stage, but will generally be less than about 60 seconds.

The post exposure bake (PEB) chill stage 330, is a stage used to control the assure that the time the substrate is at a temperature above ambient temperature is controlled so that every substrate sees the same time-temperature profile and thus process variability is minimized. Variations in the PEB process time-temperature profile can have an effect on properties of the deposited film layer and thus is often controlled to minimize process variability. The temperature of the PEB chill stage 330, is thus used to cool the substrate after the PEB stage 328 to a temperature at or near ambient temperature. The time required to complete the PEB chill stage 330 will depend on the temperature of the substrate exiting the PEB stage, but will generally be less than about 30 seconds.

The develop stage 332, is a process in which a solvent is used to cause a chemical or physical change to the exposed or unexposed photoresist and BARC layers to expose the pattern formed during the exposure process stage 326. The develop process may be a spray or immersion or puddle type process that is used to dispense the developer solvent. In some develop processes, the substrate is coated with a fluid layer, typically deionized water, prior to application of the developer solution and spun during the development process. Subsequent application of the developer solution results in uniform coating of the developer on the substrate surface. In stage 334, a rinse solution is provided to surface of the substrate, terminating the develop process. Merely by way of example, the rinse solution may be deionized water. In alternative embodiments, a rinse solution of deionized water combined with a surfactant is provided. One of ordinary skill in the art would recognize many variations, modifications, and alternatives.

In stage 336, the substrate is cooled after the develop and rinse stages 332 and 334. In stage 338, the substrate is transferred to the pod, thus completing the processing sequence. Transferring the substrate to the pod in stage 338 generally entails the process of having the front end robot 108 return the substrate to a cassette 106 resting in one of the pod assemblies 105.

In the discussion of the previous processing sequence, transfer of the substrate from various chambers of the track lithography tool 10 to other chambers was generally omitted for purposes of clarity. One of skill in the art will appreciate the use of a number of transfer robots to accomplish the various transfers between appropriate chambers.

As discussed in detail below, in accordance with embodiments of the present invention, after stage 334 or 336 a solution is applied to the wafer containing a mixture of different marker molecules. The mix may, for example, include molecules targeting the optimum critical dimension (CD) and the upper and lower control limits. Molecules of each type would have different color fluorescent tags. For purposes of the instant application, the term “critical dimension” refers to either the width of a line, or the distance between adjacent lines.

The wafer would then be rinsed to remove molecules that have not become bound to certain features present thereon. For the purposes of this invention, the term “feature” refers both to the width of feature, and to the distance between adjacent features. In certain embodiments, the surface of the wafer is maintained wet in order to prevent the marker molecules from drying on the surface. Such drying could render difficult subsequent removal of the marker molecules from the substrate surface.

The rinsed wafer is then passed under a UV lamp to excite the fluorescence, and imaged with a video camera to create a picture of regions that are properly bound. In the next stage, the wafer is rinsed in warm water to remove the marker molecules. It is then dried and returned to the pod. The following section provides a detailed discussion of such detection/measurement of a wafer utilizing marker molecules.

Critical Dimension (CD) in a semiconductor structure may be accurately measured or detected utilizing the site-specific binding properties of organic molecules or biological molecules. In accordance with one embodiment of the present invention, a fluorescent tagged organic molecule is fabricated having a distance between binding sites corresponding to the desired CD. The semiconductor device is exposed to a solution containing the organic molecule. The solution is removed and the structure is analyzed for the fluorescent tag, whose presence indicates a feature having the CD. Fluorescent tagged biological molecules of known size such as peptides or proteins, or nucleic acids such as DNA or RNA, may also be employed for CD measurement/detection purposes.

Binding between organic molecules is typically site specific. There is also an advantage if these bonds are easily made or broken, which is especially true when the bonds are relatively weak, as is the case with hydrogen bonds (as opposed to stronger ionic or covalent bonds).

Examples of such relatively weak bonds include the bonds between opposing base pairs in DNA, or bonding of polar amino acid side groups on certain proteins and associated enzymes or antibodies. Because the range of attraction is very short—at most a few angstroms, two molecules must align precisely before the bond will form. Misalignment of only a few angstroms will result in a weak bond unable to survive thermal motions. Such weak bonds are easily broken if a molecule changes conformation or is heated outside of a narrow temperature range.

Weak bond strengths, for example, allow DNA strands to be easily separated for replication, or enzymes to be detached and recycled. The polymerase chain reaction used to amplify DNA relies on this property to alternately bond and de-bond strands of DNA through heating and cooling cycles in the presence of the enzyme DNA polymerase.

In accordance with embodiments of the present invention, the binding properties of organic or biological molecules may be exploited to rapidly and accurately measure CD of a structure. Such a CD may represent the width of a feature such as a line, or the distance between adjacent features such as a pair of lines.

First, a molecule is created that is complementary to the structure to be measured. For example, if a target CD is 45+/−1 nm, then marker molecules are chosen that are complementary to 44, 45 and 46 nm structures. Each type of marker molecule would have a fluorescent tag of a different color. For example, the 44 nm molecule has a red tag, the 45 a green tag, and the 46 a yellow tag.

The wafer is washed with a solution of the molecules, and then rinsed to remove excess molecules. Illumination with a laser or UV source causes the molecules to glow, so they can be mapped after bonding to the wafer. Therefore, if the CD test sites glow green, then the CD matches the expected length and is correct. Conversely, if the CD test sites glow red the CD is too small. A yellow glow indicates the CD is too large. No glow at all indicates the CD is outside the control limits. A mix of colors may also be observed, in the event that geometries vary locally, providing a hue that may be sorted into base colors using well known image processing methods.

The molecules are then removed by washing the wafer in warm water, and may be thereafter recycled for another measurement. Alternatively, the molecules could be removed from the surface of the substrate by exposure to an enzyme or other agent causing a change in their conformality.

Many different types of molecules could be used for CD measurement/detection purposes. In one example, the molecule could be a protein having repeating polar and non-polar domains (created with polar and non-polar amino acids). The frequency of repetition of these domains would correspond to the period of the structure to be measured. Increasing the number of repeats would increase the strength of binding. A molecule having only a single domain would be suitable for measuring an isolated structure, such as a single gate.

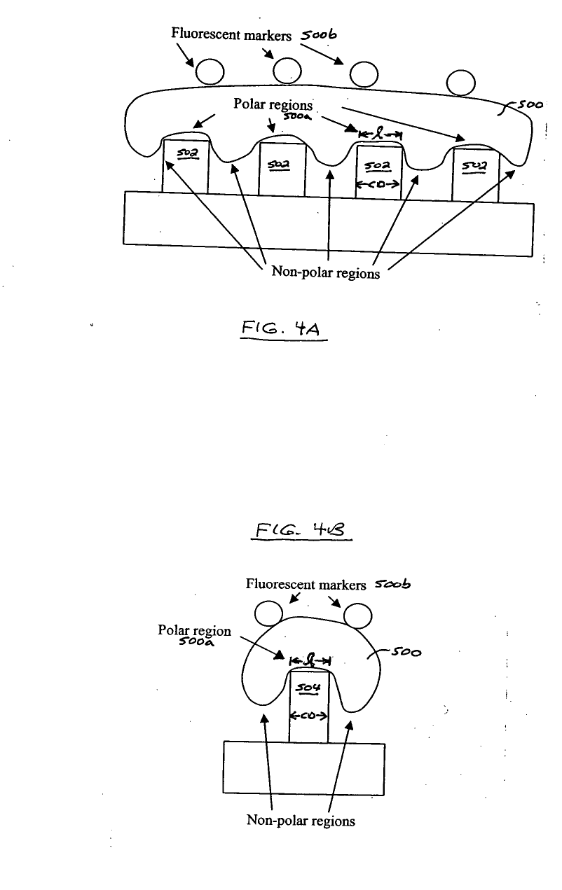

FIGS. 4A-B are simplified schematic views illustrating use of an embodiment in accordance with the present invention, wherein protein molecules 500 having fluorescent markers 500b bind to a repeating photoresist structure 502 or to a single gate structure 504. The number of fluorescent markers on a molecule will depend on the molecular structure.

In the example of the repeating structure shown in FIG. 4A, polar (hydrophilic) regions 500a of protein molecule 500 are the binding sites. These polar regions 500a are recessed, with the length l of the regions equal to the CD to be measured. Therefore, only structures with CD equal to or smaller than the width of the recesses will provide binding sites. A series of molecules with a range of widths of binding sites may be used to measure CD.

For the example of the single gate structure whose measurement is depicted in FIG. 4B, there is only one binding site 500a. Again, only gates with length equal to or smaller than the length of the opening in the binding region will provide a site for attachment to the feature.

In accordance with certain embodiments, the molecule 500 may be engineered based upon known protein or peptide structures. In accordance with alternative embodiments, molecule 500 may be found using well-known combinatorial methods whereby, for example, test structures are washed with a large number of differing molecules to identify those that adhere

In the above-referenced embodiments, binding of the marker molecule is to single or periodic structures. However, embodiments in accordance with the present invention are not limited to this particular application.

In accordance with an alternative embodiment of the present invention, a test structure may be fabricated on the wafer surface. Such a test structure may be created with a spacing of features matching the known positions of corresponding binding sites on a measurement molecule. Assuming that processing is uniform across the wafer and results in the critical dimension of the test structure matching that of active devices, marking of the test structure in accordance with embodiments of the present invention can reveal the attributes of structures patterned on the wafer surface.

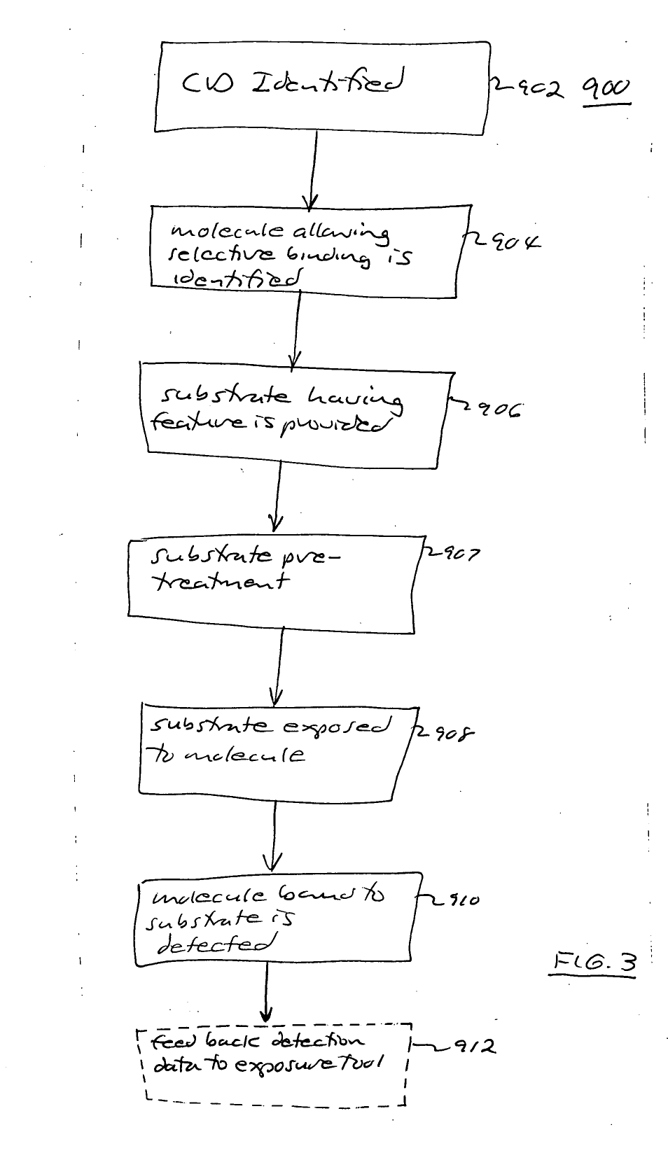

FIG. 3 shows a simplified flow chart for a method in accordance with an embodiment of the present invention. In a first stage 902 of method 900, a critical dimension of a feature on a substrate is identified.

In stage 904, a molecule having a property allowing the molecule to selectively bind to the feature is created As described in detail, the molecule features binding regions exhibiting lengths (dimensions) and/or binding properties complementary to the desired feature.

In accordance with certain embodiments, a plurality of types of marker molecules may be created exhibiting a range of lengths. Each such molecule type may have a different color marker, so that measurement can identify a range of CDs, for example an upper control limit, a lower control limit, and a target.

In stage 906, a substrate having the feature is provided. As in the embodiments previously described, the feature may comprise an actual portion of a device, interconnect, or isolation structure. In accordance with alternative embodiments, however, the feature may comprise a test structure. Such a test structure is pre-arranged to correspond to binding sites on a marker molecule. The test structure does not comprise an electrically active structure, but rather exhibits the same dimensions or properties of active devices, and is intentionally fabricated on the wafer to allow measurement/detection of those features.

Embodiments in accordance with the present invention employing measurement/detection of test structures, may offer a number advantages over the direct marking of active device structure. For example, use of a test structure would require only a portion of the substrate surface to be exposed to the marker molecules and to any excitation radiation, thereby allowing active structures (presumed to exhibit the same properties as the test structure) to be shielded from a number of stages in accordance with embodiments of the present invention. Examples of such stages include but are not limited to marker molecule exposure, rinsing, and removal stages, surface pre-treatment stages, and exposure to excitation radiation. In some cases test structures are preferred because a factory makes many different part types, each with differing features. A test structure, however, can be maintained constant, independent of the active parts being manufactured.

In optional stage 907, the substrate may be pre-treated to enhance binding with the molecule. In stage 908, the substrate is exposed to the molecule to allow it to selectively bind to the feature.

In stage 910, the molecule is detected bound to the substrate. This detection can result from the exposure of the substrate and molecules bound thereto, to excitation radiation. This excitation radiation exhibits properties (i.e. wavelength, intensity) intended to result in detectable fluorescence in the tag of the marker molecule. The excitation radiation may be produced by a variety of sources, including but not limited to ultraviolet or other types of lamps, and also radiation from scanned lasers.

In accordance with certain embodiments, an image of the substrate may be obtained, by viewing the substrate with a video camera, while exposing the substrate to radiation to excite the fluorescent tag.. As shown in optional stage 912, an image of the map of the substrate bearing excited marker molecules may then be fed back to a processing tool for analysis, for example allowing an exposure tool to correct for regions of improper development, or an etching tool to correct for regions of incomplete etching.

The marker molecules may be removed from the wafer using, for example, a rinse in warm water. The wafer is then dried before exiting the track system.

While the above embodiments have discussed the use of peptides or proteins as molecules for measuring CD, the present invention is not limited to this particular application. Other embodiments would fall within the scope of the instant application.

For example, nucleic acid molecules such as DNA or RNA can alternatively be employed for CD measurement. In accordance with one such embodiment, a strand of DNA may be constructed having alternating regions comprised exclusively of one base or base type.

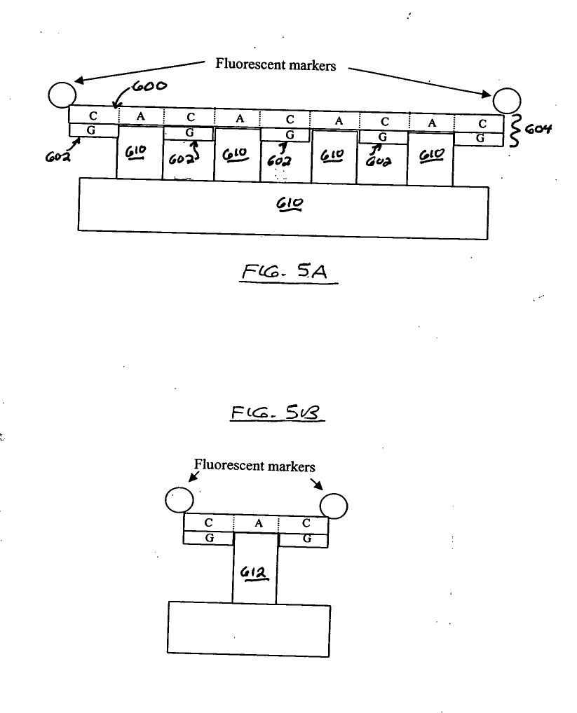

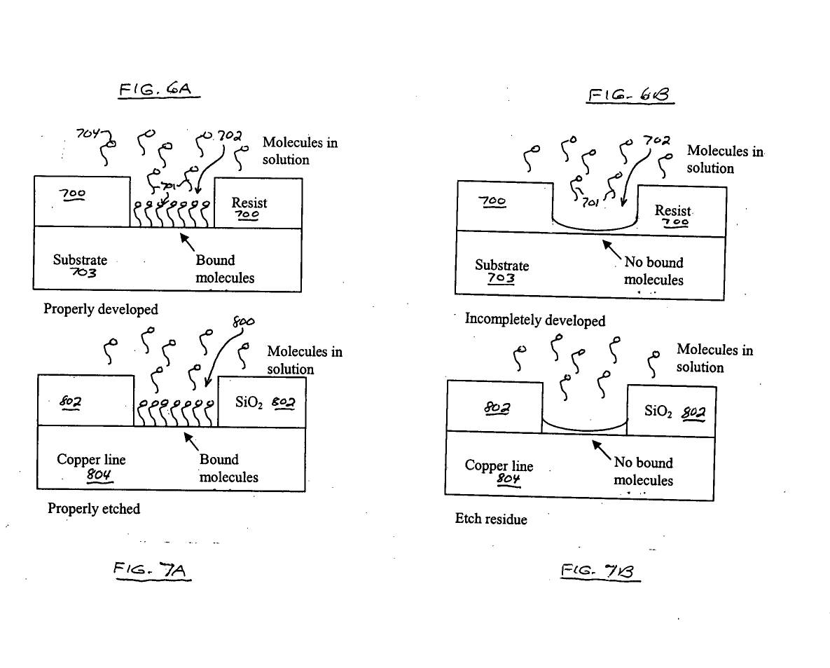

FIG. 5A shows the use of such a DNA molecule for measurement of CD's of a repeating structure 610. FIG. 5B shows the use of a DNA molecule for measurement of CD of a single structure 612. In the case shown in FIG. 6A, these repeating base regions are made of cytosine (C) and adenine (A). The length of the A regions is equal to the feature size, and the length of the C regions is equal to the gaps between features.

Length of the structures of the nucleic acid molecule can be engineered to the accuracy of a single base, 0.34 nm. In this example, A is used as the open base because, as a purine, it has three hydrogen bonding sites (versus two for a pyrimidine). Next, the single DNA strand 600 having the desired structure is hybridized with short single strands 602 of guanine (G) equal in length to the C regions. Combination of the single strands 600 and 602 yields a double-stranded DNA molecular structure 604 having only the A regions free to bond with a corresponding feature on the wafer or substrate. Double-stranded DNA structure 604 will then preferentially bond to a repeating structure when the width of the features is equal to or less than the length of the A regions. In accordance with other embodiments, C-G pairs of nucleotides may be replaced with A-T pairs, using C or G as the exposed nucleotide. In accordance with still other embodiments, nucleotides T or U (uracil) may be exposed.

To enhance such preferential bonding, the photoresist could be chemically modified, for instance, by being hydrogenated, by being mixed with another chemical that increases the availability of hydrogen bonds at the photoresist surface, or by application of an adhesion promoter. As in the earlier example, the DNA is tagged with phosphors to allow identification under UV or laser illumination. Nucleic acid strands having A regions of different lengths covering the CD range of interest, are mixed together in the solution applied to the wafer. In accordance with still other embodiments, C, T or U may be the exposed nucleotide, as described above.

FIGS. 5A-B are simplified in that they do not depict the helical nature of the DNA molecule, especially in the C-G regions. Consequently, some lengths will not present binding sites in the correct orientation. However, this should average over a number of features, preserving the selectivity of the bonding.

It should be noted that the strength and specificity of bonding can be adjusted by a number of factors, such as choice of the length of the strand, and/or whether a purine or pyrmidine is used as the open base and surface preparation of the feature. The temperature may also be adjusted to control bonding; with a greater number of bonding sites required to hold the molecule in place at higher temperatures.

For the case of the isolated feature 612 shown in FIG. 5B, only a single opening is made. In one embodiment, the opening with is a equal to or greater than the desired CD, but this is not required. A range of molecules with different opening widths covering the desired CD range are mixed together in the solution applied to the wafer. It may be necessary to apply an adhesion promoter that adds hydrogen bonding sites to the top of the features. As with the proteins, the DNA is washed off in warm water, which causes the hydrogen bonds to break. After cooling the DNA molecules are available for re-use.

One potentially important advantage offered by the use of nucleic acids for measurement purposes, is the relative ease and low cost of manufacturing these molecules. Specifically, because nucleic acid molecules can readily be amplified utilizing at least the well known polymerase chain reaction (PCR) technique, it is possible to produce tailored nucleic acids in large volumes. As a result of this advantage, nucleic acids such as DNA may ultimately prove less expensive to produce than other molecules, such as proteins.

It should be noted that other variations in the method are possible. For example, while the above-referenced figures illustrate embodiments utilizing binding affinity to detect CD of raised features, this is not required by the present invention. In accordance with other embodiments, organic or biological molecules could alternatively be employed to measure a dimension of a recess such as a via hole. In accordance with such an embodiment, a molecule may be chosen that has a particular size, so that it will not fit in the hole if the hole is too small.

In accordance with still further alternative embodiments of the present invention, a particular molecule employed for measurement purposes may be designed to preferentially adhere to one material of interest over another material. For example, the molecule may be designed to adhere to copper oxide (a thin layer of which is typically present over copper after exposure to air), but not to photoresist. In certain embodiments, the marker molecule or an additive may convert copper oxide to copper, with the marker molecule substituting for the oxygen.

In accordance with still another embodiment of the present invention, the measuring/marking molecule may adhere to silicon oxide but not photoresist. In accordance with still another embodiment, the measuring molecule may adhere to copper, but not to silicon dioxide. The selective adhesion by the marker molecule may arise through hydrogen bonds, which are weak and allow the molecule to be readily removed from the measured feature, for example by rinsing in warm water.

In one specific example, a molecule exhibiting preferential binding characteristics may be employed to determine whether photoresist has been fully developed. FIGS. 6A-B which show simplified schematic views of such an embodiment. In particular FIG. 6A shows a view in which photoresist 700 has properly been fully developed, and FIG. 6B shows a view in which photoresist 700 is incompletely developed.

A solution containing molecule 701 is spun on a wafer following photoresist development. This photoresist development removes the photoresist from the underlying layer 703, which may, for example, comprise silicon dioxide. If this development stage is complete, the expected condition is exposure of silicon dioxide 703 molecule 701 will stick to the bottom of the via hole 702. If this development stage is incomplete, resist remaining at the bottom of the hole will prevent the measuring molecule from sticking.

As shown in the previous figures, the marking molecule 701 has a fluorescent tag 704, so illuminating the wafer under a UV lamp or laser scanning and imaging will map the wafer for areas of incomplete photoresist development. If the features are entirely covered with photoresist, there will be a lack of fluorescence. Partial development will appear as a reduced level of fluorescence. After mapping, the marker molecules may be washed off in warm water.

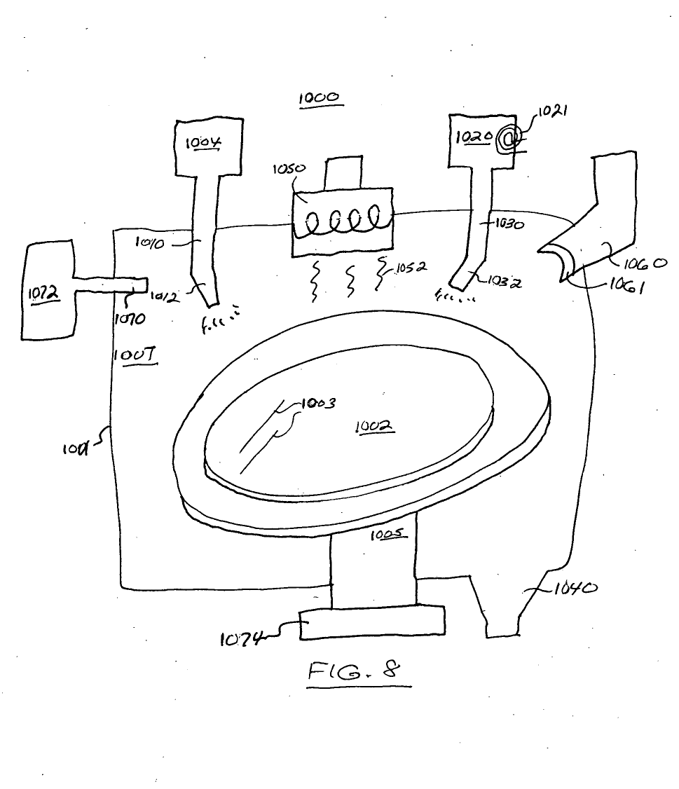

A second example is shown in the simplified schematic views of FIGS. 7A-B. In this embodiment, via hole 800 is etched through an interlayer dielectric material 802 such as silicon dioxide or Black Diamond™ (BD-II) to underlying copper line 804.

In this case, the marking molecules selectively bond to copper. A completed via etching stage should result in the expected condition of the removal of copper. However, the presence of any residue following this etching stage, even a monolayer, will prevent the marker molecules from being exposed to the copper surface, preventing the weak bonds from forming and interfering with binding.

In this particular embodiment, the detecting stage is performed in a clean stage that routinely follows the etching stage. After the etching, the wafer is brought to a cleaner. The cleaning stage is a wet process anyway, and therefore the marking stage in accordance with an embodiment of the present invention may readily be integrated. Specifically, the cleaner typically has several chemical baths. In this case, one bath would include the marker molecules. The wafer is exposed to the markers, rinsed to remove excess marker molecules, and then mapped to reveal areas of incomplete etching. The marker is then removed as part of the conventional cleaning process.

A variety of techniques may be utilized to map the wafer. In accordance with one embodiment, the wafer may be mapped by observation with a video camera while under ultraviolet illumination. In accordance with an alternative embodiment of the present invention, the wafer may be scanned with a laser to excite the fluorescence and be viewed with a video camera.

FIG. 8 shows a simplified schematic view of an embodiment of an apparatus which may be utilized to detect wafer properties in accordance with the present invention. Detection module 1000 comprises walls 1001 housing chamber 1007 enclosing pedestal 1005 supporting substrate 1002 having features 1003 patterned thereon. Reservoir 1004 holds the solution containing the marker molecule, and is in fluid communication with the chamber through inlet 1010 and nozzle 1012. Once the wafer is present in the chamber, the solution comprising the marking molecule is applied to its surface.

Reservoir 1020 holds a diluent such as water, and is in fluid communication with the chamber through inlet 1030 and nozzle 1032. Water from reservoir 1020 may be sprayed on the wafer surface prior to, during, and after application of the solution containing the marker molecule.

Prior to application of the solution containing the marker molecule, the water from reservoir 1020 may be applied to wet the surface of the substrate, preparing it to receive the marker molecule. During application of the solution containing the marker molecule, the water from reservoir 1020 may be applied to maintain the wetness of the wafer surface, allowing bonds to form between the features and the marker molecule.

Subsequent to exposure of the wafer to the marker molecule and detection of binding, the water from reservoir 1020 may be applied to rinse and remove the marker molecule from the surface of the wafer. Accordingly, reservoir 1020 may be in thermal communication with a heat source 1021 such as a heater coil, which can increase the temperature of the water. The elevated temperature of heated water applied from reservoir 1020 may weaken bonds between the marker molecule and the feature, thereby facilitating removal of the marker molecule. Unwanted material in the chamber may be removed through drain 1040.

Radiation source 1050 is in electromagnetic communication with the interior of the chamber, and specifically is configured to irradiate the substrate with excitation radiation. Excitation radiation 1052 emitted from source 1050 impinges upon the marker molecule, allowing the marker molecule to be sensed by detector 1060 that is also in electromagnetic communication with the chamber. The source could be, for example, an ultraviolet light or a laser. Detector 1060 may comprise a camera configured to sense fluorescence, or may comprise a device configured to perform Raman spectroscopy. Typically, detector 1060 would be equipped with one or more filters 1061 to filter out the excitation radiation and background light, allowing sensitive detection of fluorescence or other types of radiation indicating the presence of the marker molecule.

In accordance with certain embodiments the detection module may be combined with a photoresist developer module. Thus as shown in FIG. 8, the chamber would also include an inlet 1070 in fluid communication with a reservoir 1072 containing developer solution, and a motor 1074 configured to rotate the substrate support to spin the developer solution onto the wafer. Such an embodiment could also include a device for irradiating only selected portions of the wafer surface in order to develop and pattern the resist material, and particular embodiments could utilize the same radiation source for marker excitation and resist development.

The previous description has focused upon particular embodiments, but variations on those embodiments also fall within the scope of the present invention. For example, while the above discussion has focused upon use of maker molecules having fluorescent tags, this is not required. In accordance with alternative embodiments, the marker molecule itself may fluoresce, obviating the need to use a separate fluorescent tag. Such an embodiment however, would provide only a single color to indicate, for example, that the CD is either correct or incorrect.

Moreover, while the previous discussion has relied upon fluorescence to detect critical dimension or other properties of a substrate, the present invention is also not limited to this specific type of detection. In accordance with alternative embodiments, alternative detection techniques could be employed. For example, a marker molecule could include different metals or side chains that are detectable utilizing techniques such as Raman spectroscopy, and still fall within the scope of the present invention.

Furthermore, other alternative embodiments of detection/measurement methods could combine or separate stages, and still remain within the scope of the present invention. For example, resist development stages are typically terminated by rinsing the wafer. In accordance with embodiments of the present invention, the rinsing stage to terminate resist development, could also include the introduction of marker molecules to detect the degree of completeness of this development stage.

The above discussion has focused upon the use of molecular markers to reveal information about features formed on a semiconductor workpiece. Embodiments in accordance with the present invention are not limited to this particular application, however. Embodiments in accordance with the present invention may be utilized to identify features on other types of workpieces, including but not limited to glass LCD panels, magnetic disk media, or other types of substrates.

The examples and embodiments described herein are for illustrative purposes only. Various modifications or changes in light thereof will be suggested to persons skilled in the art and are to be included within the spirit and purview of this application and scope of the appended claims. It is not intended that the invention be limited, except as indicated by the appended claims.

What is claimed is:

1. A method for providing information regarding a feature on a substrate, the method comprising:
providing a substrate having a feature;
exposing the substrate to at least one molecule selectively binding to the feature; and
detecting the molecule bound to the feature.

2. The method of claim 1 wherein the molecule exhibits a length corresponding to a critical dimension (CD) of the feature.

3. The method of claim 2 wherein the length corresponds to a domain comprising one of a hydrophilic region, a hydrophobic region, and a hydrogen bonding region.

4. The method of claim 3 wherein the molecule comprises a nucleic acid and the domain comprises one of a pyrimidine region and purine region.

5. The method of claim 1 wherein the molecule exhibits a length one of greater than and smaller than a critical dimension (CD), the method further comprising:
exposing the substrate to a second molecule exhibiting a length corresponding to the critical dimension; and
the detecting further comprises detecting an absence of the second molecule bound to the feature.

6. The method of claim 5 wherein:
the molecule includes a first fluorescent tag and the second molecule includes a second fluorescent tag, the method further comprising; and
the detecting comprises illuminating the substrate with radiation to trigger fluorescence of the first tag.

7. The method of claim 1 wherein the molecule comprises one of a peptide and a protein.

8. The method of claim 1 wherein the molecule exhibits a preferred binding affinity to an expected characteristic of the feature.

9. The method of claim 8 wherein the expected characteristic is lacking if fabrication of the feature is incomplete.

10. The method of claim 1 further comprising modifying a surface property of the substrate prior to exposure to the molecules.

11. The method of claim 1 further comprising removing the molecules from the surface after the detecting.

12. The method of claim 1 further comprising removing excess quantities of the molecule prior the detecting.

13. The method of claim 1 further comprising controlling temperature during the exposing to adjust a binding strength between the molecule and the feature.

14. The method of claim 1 wherein a size of the molecule allows it to fit within features larger than one of a predetermined size and shape.

15. The method of claim 1 wherein the molecule comprises a fluorescent tag, and the method further comprises exposing the substrate to ultraviolet light to excite the fluorescent tag.

16. The method of claim 1 wherein the molecule comprises a fluorescent tag, and the method further comprises exposing the substrate to laser light to excite the fluorescent tag.

17. The method of claim 1 wherein the molecule comprises a fluorescent tag, and the method further comprises obtaining an image of the substrate by viewing the substrate with a video camera while exposing the substrate to radiation to excite the fluorescent tag..

18. The method of claim 17 further comprising:
interpreting the image to obtain a map of the CD over the full wafer;
feeding back the map to a processing tool.

19. The method of claim 1 wherein the molecule is detected bound to one of a test feature and a device structure.

20. The method of claim 1 wherein the molecule includes an element detectable through Raman spectroscopy, and the detecting comprises performing Raman spectroscopy on the substrate.

21. The method of claim 1 wherein the molecule is fluorescent, and the detecting comprises irradiating the substrate with radiation to cause fluorescence.

22. A composition for indicating conformity of a substrate feature to a critical dimension (CD), the composition comprising:
a solvent; and
a molecule having a marker, a length corresponding to a critical dimension, and a site exhibiting binding affinity with the feature.

23. The composition of claim 22 wherein the site is selected from the group consisting of a pyrimidine region, a purine region, a hydrophobic region, a hydrophilic region, and a hydrogen bonding region.

24. The composition of claim 22 further comprising a second molecule having a second marker different from the first marker, and a second length corresponding to a distance that is one of larger than and smaller than the critical dimension.

25. The composition of claim 22 wherein the marker comprises a fluorescent group.

26. An apparatus for detecting a feature on a semiconductor workpiece, the apparatus comprising:
walls enclosing a chamber housing a substrate support and a drain;
a fluid inlet configured to receive at least one of diluent from a first reservoir and a marker molecule solution from a second reservoir;
a radiation source in electromagnetic communication with the chamber and configured to irradiate a substrate positioned on the support with excitation radiation; and
a detector in electromagnetic communication with the chamber through a filter, the detector configured to sense radiation emitted from the substrate in response to exposure to the excitation radiation.

27. The apparatus of claim 26 wherein the radiation source is selected from the group comprising a lamp and a laser.

28. The apparatus of claim 26 further comprising:
a second fluid inlet configured to receive a developer solution from a third reservoir; and
a motor configured to rotate the support to spin the developer solution onto the substrate.

29. The apparatus of claim 26 wherein the filter is configured to filter the excitation radiation.

30. The apparatus of claim 26 wherein the detector comprises a camera configured to detect fluorescence.
